(19) United States
(12) Patent Application Publication
(10) Pub. No.: US 2018/0125458 A1
(43) Pub. Date: May 10, 2018

(54) ULTRASONIC IMAGE GENERATION SYSTEM AND ULTRASONIC WIRELESS PROBE

(71) Applicant: SOCIONEXT INC., Yokohama-shi (JP)

(72) Inventors: Hiroaki TAKAGI, Yokohama-shi (JP); Naoto YONEDA, Yokohama-shi (JP); Naoto ADACHI, Yokohama-shi (JP); Masaya TAMAMURA, Yokohama-shi (JP); Amane INOUE, Yokohama-shi (JP)

(21) Appl. No.: 15/863,488

(22) Filed: Jan. 5, 2018

Related U.S. Application Data
(63) Continuation of application No. PCT/JP2016/056113, filed on Feb. 29, 2016.

(30) Foreign Application Priority Data
Jul. 9, 2015 (JP) 2015-137618

(51) Int. Cl. A61B 8/08; A61B 8/00

(52) U.S. Cl. CPC A61B 8/5207; A61B 8/54; A61B 8/4472

(57) ABSTRACT

An ultrasonic image generation system has a probe having an ultrasonic transducer configured to transmit and receive an ultrasonic signal, a processor configured to generate an ultrasonic image signal by processing a received signal of the ultrasonic transducer as well as to generate a drive signal that is supplied to the ultrasonic transducer, and a probe-side wireless communicator; and a terminal having a terminal-side wireless communicator configured to wirelessly communicate with the probe-side wireless communicator, a display configured to display an ultrasonic image based on the ultrasonic image signal, and an operation panel configured to input general measurement information, wherein the probe has a controller configured to determine control information necessary for generation of the drive signal and processing of the received signal from the general measurement information transmitted from the terminal.

### CROSS-REFERENCE TO RELATED APPLICATION

This application is a continuation application of International Application PCT/JP2016/056113 filed on Feb. 29, 2016, the entire contents of which are incorporated herein by reference.

### FIELD

The present invention relates to an ultrasonic image generation system and an ultrasonic wireless probe.

### BACKGROUND

An ultrasonic image generation system that generates an ultrasonic image representing a state of the inside of a living body by irradiating the living body with an ultrasonic wave and detecting the reflected wave is widely used. A common ultrasonic image generation system has a main body unit and an ultrasonic transducer (hereinafter, referred to as an ultrasonic unit) connected to the main body unit by a cable. The main body unit generates a drive signal of the ultrasonic unit and transmits the generated drive signal to the ultrasonic unit via the cable. The ultrasonic unit outputs an ultrasonic wave in accordance with the drive signal, generates a reflected ultrasonic wave signal by capturing the reflected ultrasonic wave, and transmits the reflected ultrasonic wave signal to the main body unit. The main body unit generates an ultrasonic image by processing the received reflected ultrasonic wave signal and displays the ultrasonic image on a display.

In recent years, an ultrasonic image generation system is expected to be turned into a mobile device and reduction in size, reduction in cost, and improvement of operability have been sought. The ultrasonic unit, which is grasped by the hand and contacts with a living body portion, is connected to the main body unit by a cable, and therefore the operation thereof is restrained. Thus, it has been proposed to improve operability by eliminating the cable and carried out data communication by wireless communication, in other words, by turning data communication into wireless communication. However, since the amount of data to be transferred in the communication between the ultrasonic unit and the main body unit is large, it is difficult to communicate a large amount of data in a short time by wireless communication, to reduce the size of the ultrasonic image generation system and to improve operability of the ultrasonic image generation system.

Further, in general, the ultrasonic image generation system is a dedicated device and the cost is high. Thus, it is desired to reduce the cost by making use of a general-purpose terminal, such as a PC, a PC tablet, and a smartphone. However, a dedicated device aims at improvement of operability by providing a lot of mechanical knobs and switches, and therefore there has been such a problem that it is difficult to implement the same operability as that of a dedicated device by a general-purpose terminal. Further, since the cable is compatible with a special I/F, the ultrasonic image generation system is a dedicated terminal.

### RELATED DOCUMENTS

[Patent Document 1] Japanese Laid Open Patent Document No. 2012-187244

[Patent Document 2] Japanese Laid Open Patent Document No. 2010-57562

[Patent Document 3] U.S. Patent Application No. 2003/0139664

### SUMMARY

An ultrasonic image generation system of a first aspect has a probe unit and a terminal. The probe unit has an ultrasonic unit configured to transmit and receive an ultrasonic signal, a drive control/signal processing unit configured to generate an ultrasonic image signal by processing a received signal of the ultrasonic unit as well as to generate a drive signal that is supplied to the ultrasonic unit, and a probe-side wireless communication unit. The terminal has a terminal-side wireless communication unit configured to wirelessly communicate with the probe-side wireless communication unit, a display unit configured to display an ultrasonic image based on the ultrasonic image signal, and an operation unit configured to input general measurement information. The probe unit has a control information determination unit configured to determine control information necessary for the generation of a drive signal and the processing of a received signal from general measurement information transmitted from the terminal.

The object and advantages of the embodiments will be realized and attained by means of the elements and combination particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating a configuration of a common ultrasonic image generation system.

FIG. 2 is a block diagram illustrating a configuration of an ultrasonic image generation system of an embodiment.

FIG. 3 is a diagram illustrating a display example of a display unit of a general-purpose terminal.

FIG. 4 is a diagram illustrating a parameter setting table of general measurement information and control information.

### DESCRIPTION OF EMBODIMENTS

Before explaining an ultrasonic image generation system of an embodiment, a common ultrasonic image generation system is explained.

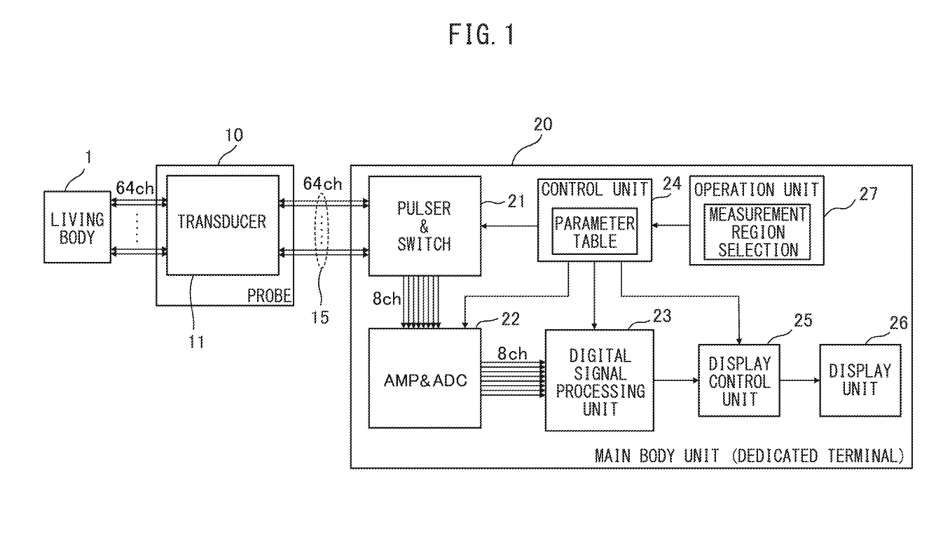

FIG. 1 is a block diagram illustrating a configuration of a common ultrasonic image generation system.

The common ultrasonic image generation system has a probe 10, a main body unit 20, and a cable 15 that connects the probe 10 and the main body unit 20. The probe 10 and the main body unit 20 are connected by the cable 15 and communication is performed between the main body unit 20 and the probe 10 and at the same time, a power source is supplied from the main body unit 20 to the probe 10.

The probe 10 is held by a measurer of the ultrasonic image generation system and is in contact with the surface of a living body 1, which is the target of measurement, and thereby, the inside of the living body 1 is measured by ultrasonic waves. The probe 10 has a transducer 11 that converts a high-voltage pulse signal received via the cable into a sound wave, outputs the sound wave to the living body 1, and converts the sound wave reflected at the boundary between muscle and fat, whose acoustic impedances are different, within the living body 1 into an electric signal.

The main body unit 20 has a pulser & switch 21, an AMP & ADC 22, a digital signal processing unit 23, a control unit 24, a display control unit 25, a display unit 26, and an operation unit 27. The pulser & switch 21 generates a high-voltage pulse signal that is supplied to the transducer 11, and selects the electric signal of the reflected sound wave by a switch circuit and outputs the electric signal to the AMP & ADC 22. The pulser & switch circuit 21 performs, in response to the control signal from the control unit 24, processing to bring into focus the signals transmitted simultaneously in a plurality of channels within the living body by changing the amount of delay for each channel. After amplifying the electric signal by the amplifier (AMP) in response to the control signal from the control unit 24, the AMP & ADC 22 converts the electric signal into a digital signal by the ADC (Analog-to-Digital Converter) and outputs the digital signal to the digital signal processing unit 23. Here, an example is illustrated, in which the number of input/output channels of the transducer 11 is sixty-four and the number of channels of the AMP & ADC 22 is eight, but these numbers are optional and the width and resolution of an ultrasonic image that is obtained are determined by the channel interval and the number of channels of the transducer 11. The pulser & switch 12 performs simultaneous pulser transmission corresponding to the number of channels of the ADC.

The digital signal processing unit 23 converts the digital signal from the AMP & ADC 22 into luminance information by making use of the control signal from the control unit 24, and performs gain correction or the like that takes into consideration the attenuation within the living body and generates an ultrasonic image signal. By the above processing, an ultrasonic image signal can be obtained by carried out transmission and reception while shifting the sixty-four channels one by one and processing the received signals. The display control unit 25 receives an ultrasonic image signal from the digital signal processing unit 23 and performs control so as to display an ultrasonic image on the display unit 26.

The control unit 24 receives general measurement information, which is input by a measurer making use of the operation unit 27, for example, measurement regions, sex, age, height, weight (or BMI), and so on, of a subject of measurement. The operation unit 27 may be a touch panel. The control unit 24 has a parameter table and determines control information suitable to the general measurement information by making use of the table. The control information includes gain, focus position, number of focus points, image processing pattern to be used, and so on. The control unit 24 controls the pulser & switch circuit 21, the AMP & ADC 22, the digital signal processing unit 23, and the display control unit 25 based on the control information. As described previously, the pulser & switch circuit 21 performs the processing to bring into focus the signals transmitted simultaneously in a plurality of channels within the living body by changing the amount of delay for each channel, and the focus position and the number of focus points are used for the control by the control unit 24. Further, the gain is used for gain adjustment in the AMP & ADC 22 and the image processing pattern is used for determination of an image processing method that is used in the digital signal processing unit 23. Furthermore, the control information is used by the display control unit 25 in order to display an appropriate ultrasonic image on the display unit 26. The general measurement information is supplied to the display control unit 25 and displayed on the display unit 26 to check a subject of measurement.

The configuration of the above-described common ultrasonic image generation system is widely known, and therefore further explanation is omitted.

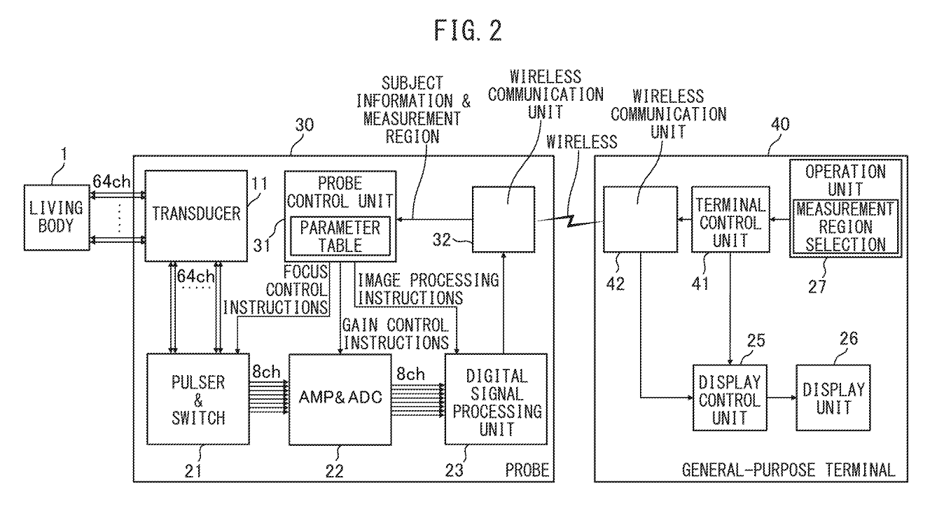

FIG. 2 is a block diagram illustrating a configuration of an ultrasonic image generation system of the embodiment.

The ultrasonic image generation system of the embodiment differs from the common apparatus in FIG. 1 in that a general-purpose terminal 40 is employed in place of the main body unit 20, that part of the components of the main body unit 20 are moved to the probe side, and that the general-purpose terminal 40 and the probe are connected by wireless communication, not by a cable. Thus, the components in common between FIG. 1 and FIG. 2 are indicated by attaching the same reference symbols and explanation thereof is omitted.

The ultrasonic image generation system of the embodiment has a probe 30 and the general-purpose terminal 40. The probe 30 and the general-purpose terminal 40 are capable of communication by wireless communication. Because no cable is provided, a power source is not supplied to the probe 30, and therefore the probe 30 has a battery and each unit is driven by a battery. The battery may be a primary battery or a rechargeable secondary battery.

The probe 30 has a probe control unit 31 and a wireless communication unit 32, in addition to the transducer 11, the pulser & switch 21, the AMP & ADC 22, and the digital signal processing unit 23. The general-purpose terminal 40 has a terminal control unit 41 and a wireless communication unit 42, in addition to the display control unit 25, the display unit 26, and the operation unit 27. The function of the control unit 24 in FIG. 1 is implemented by the probe control unit 31 and the terminal control unit 41. The parameter table to determine control information suitable to the general measurement information is provided to the probe control unit 31. The terminal control unit 41 only performs simple processing in relation to the input of general measurement information.

The wireless communication unit 32 and the wireless communication unit 42 each have a short-range wireless communication function (for example, BLUETOOTH (registered trademark)) and are capable of wirelessly communicating with each other (wireless communication). No cable is connected to the probe as described above, and therefore operability improves.

As the general-purpose terminal 40, any terminal can be used as long as having a display function, an input function, and a wireless communication function and for example, the terminal is used by installing application software for an ultrasonic image generation system to a PC tablet, a PC, a smartphone, and so on.

As above, in the ultrasonic image generation system of the embodiment, in order to implement wireless communication, first, the pulser & switch 21, which is the function on the side of the main body in the conventional system, is arranged on the side of the probe 30. Thus, the high-voltage pulse signal between the transducer and the pulser & switch, which is conventionally transferred by a cable, and the data of the reflected wave from the living body are transferred on the same substrate, and therefore a cable may be eliminated. Further, by arranging the AMP & ADC 22 and the digital signal processing unit 23 on the side of the probe side 30, the data of the reflected wave may be turned into image data by digital signal processing on the side of the probe 30. Thus, the size of data that is communicated is compressed, and therefore the data may be wirelessly transferred to the general-purpose terminal 40 with ease.

The pulser & switch 21, the AMP & ADC 22, and the digital signal processing unit 23 are arranged on the side of the probe 30 and only the already-existing functions of the general-purpose, such as the display function, the input function, and the wireless communication function, are used, and therefore the general-purpose terminal 40 may be implemented by general-purpose components. Thus, the cost and size of the apparatus may be reduced.

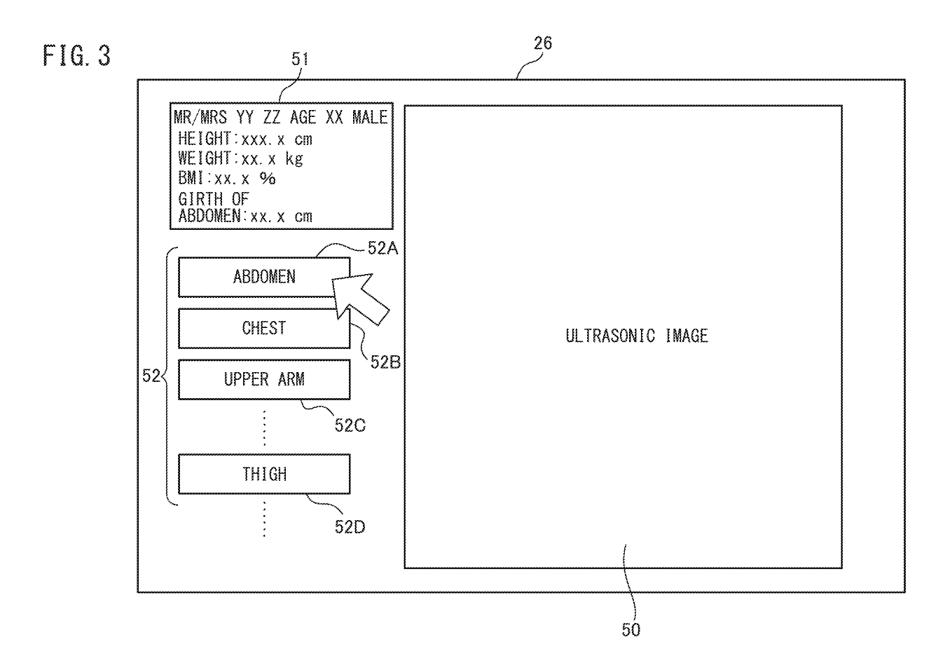

FIG. 3 is a diagram illustrating a display example of the display unit of the general-purpose terminal.

On the display unit 26 of the general-purpose terminal 40, an acquired ultrasonic image 50, subject information 51, such as name, age, sex, height, weight, BMI (Body Mass Index), and girth of abdomen of a subject, and measurement-target regions 52 (abdomen 52A, chest 52B, upper arm 52C, thigh 52D, and so on) are displayed. The subject information 51 and the measurement-target regions 52 are input by a measurer by making use of the operation unit 27 while checking the display contents on the display unit 26. However, when a general-purpose terminal compatible with the measurer is used, information stored in advance in the general-purpose terminal may be used. Further, when the display unit 26 is a touch panel which has a touch screen function, the operation unit 27 is not necessary and the information may be input by touching the display unit 26.

The terminal control unit 41 transmits the input measurement regions and subject information (may be only the information necessary for measurement) to the probe control unit 31 via the wireless communication unit 42 and the wireless communication unit 32. The probe control unit 31 determines control information in accordance with the parameter table based on the received information and controls the pulser & switch 21, the AMP & ADC 22, and the digital signal processing unit 23. Thus, the load to set parameters in the general-purpose terminal 40 is reduced, and operability may be maintained.

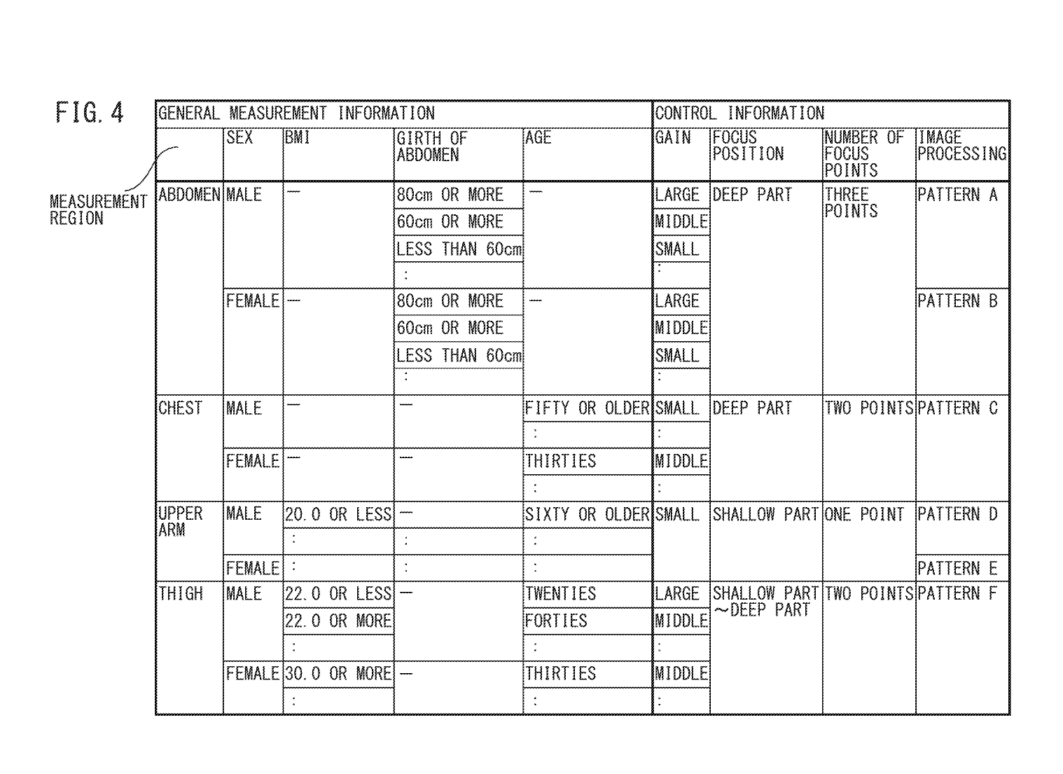

FIG. 4 is a diagram illustrating a parameter setting table of general measurement information and control information. The general measurement information includes measurement regions (abdomen, chest, upper arm, thigh, and so on), sex, BMI (weight/(height)²), and age, and the control information includes gain, focus position, number of focus points, and image processing pattern.

For example, when the girth of the abdomen of a subject is large, the depth of measurement of the abdomen is greater, and therefore it is necessary to appropriately adjust the image luminance by increasing the gain of the reflected wave data at the deep part by taking into consideration the attenuation within the body and an appropriate gain is set to the table. Further, the focus point position changes in accordance with the depth of measurement, and therefore it is necessary to change the amount of delay of the pulser & switch circuit 21 and it is supposed to improve image quality by providing a plurality of focus points in accordance with the depth. Thus, an appropriate focus position and an appropriate number of focus points are set to the table. In addition to the above, it is considered to change the measurement range for each measurement region or to change the image processing pattern of the filter processing and the like in accordance with an image with features, and therefore an appropriate image processing pattern is set to the table for each measurement region.

In the embodiment, the side of the probe 30, not the side of the general-purpose terminal 40, is caused to have the table of the image optimization parameters, and therefore it is only required to update the table information on the probe at the time of updating the table information, and no task is required for the side of the general-purpose terminal 40. Thus, measurement may be carried out with the same parameters also when a different general-purpose terminal is used.

As above, the embodiment is explained, but all the examples and conditions described here are described for the purpose of aiding understanding of the invention and the concepts of the invention to be applied to the art. Particularly, the examples and conditions described are not intended to limit the scope of the invention and the configurations of such examples in the specification do not indicate the superiority and inferiority of the invention. The embodiment of the invention is described in detail, but it should be understood that the various changes, substitutions, and alterations can be made without departing the sprit and scope of the invention.

### CITATION LIST

11 transducer
21 pulser & switch
22 AMP & ADC
23 digital signal processing unit
25 display control unit
26 display unit
27 operation unit
30 probe
31 probe control unit
32 wireless communication unit
40 general-purpose terminal
41 terminal control unit
42 wireless communication unit

What is claimed is:

1. An ultrasonic image generation system comprising:
a probe including an ultrasonic transducer configured to transmit and receive an ultrasonic signal, a processor configured to generate an ultrasonic image signal by processing a received signal of the ultrasonic transducer as well as to generate a drive signal that is supplied to the ultrasonic transducer, and a probe-side wireless communicator; and
a terminal including a terminal-side wireless communicator configured to wirelessly communicate with the probe-side wireless communicator, a display configured to display an ultrasonic image based on the ultrasonic image signal, and an operation panel configured to input general measurement information, wherein
the probe includes a controller configured to determine control information necessary for generation of the drive signal and processing of the received signal from the general measurement information transmitted from the terminal.

2. The ultrasonic image generation system according to claim 1, wherein
the controller includes a table in which the general measurement information and the control information are associated with each other.

3. The ultrasonic image generation system according to claim 1, wherein
the general measurement information includes measurement-target region, sex, and BMI, and
the control information includes gain, focus position, number of focus points, and image processing pattern.

4. The ultrasonic image generation system according to claim 2, wherein
the general measurement information includes measurement-target region, sex, and BMI, and
the control information includes gain, focus position, number of focus points, and image processing pattern.

5. An ultrasonic wireless probe comprising:
an ultrasonic transducer configured to transmit and receive an ultrasonic signal;
a processor configured to generate an ultrasonic image signal by processing a received signal of the ultrasonic transducer as well as to generate a drive signal that is supplied to the ultrasonic transducer;
a wireless communicator; and
a controller configured to determine control information necessary for generation of the drive signal and processing of the received signal from general measurement information received by the wireless communicator, wherein
the ultrasonic image signal is transmitted from the wireless communicator.
